(19) United States
(12) Patent Application Publication
(10) Pub. No.: US 2008/0300314 A1
(43) Pub. Date: Dec. 4, 2008

(54) Cooling Compounds

(75) Inventors: Christophe C. Galopin, Chesterfield, VA (US); Pablo Victor Krawec, Cincinnati, OH (US); Jay Patrick Slack, Loveland, OH (US); Lori W. Tigani, Salisbury, MD (US); Stefan Michael Furrer, Cincinnati, OH (US)

Correspondence Address: CURATOLO SIDOTI CO., LPA, 24500 CENTER RIDGE ROAD, SUITE 280, CLEVELAND, OH, 44145, US

(73) Assignee: Givaudan SA, Vernier (CH)

(21) Appl. No.: 12/165,202

(22) Filed: Jun. 30, 2008

Related U.S. Application Data
(63) Continuation-in-part of application No. 11/437,294, filed on May 19, 2006, now Pat. No. 7,414,152.
Application No. 11/437,294 is a continuation-in-part of application No. PCT/CH2004/000646, filed on Oct. 28, 2004.
(60) Provisional application No. 60/523,977, filed on Nov. 21, 2003.

(51) Int. Cl. A61K 8/42; C07C 255/20; C07C 235/32; A61Q 19/00

(52) U.S. Cl. 514/621; 564/170; 564/161; 514/622; 558/410

(57) ABSTRACT

Compounds of the formula I give a cooling sensation to the mouth and skin:

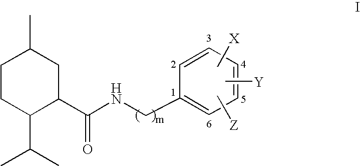

In Formula I, m is 0 or 1; X is —(CH₂)ₙ—R, where n is 0 or 1 and R is selected from —OR′. —COOH, —COOR″, —SO₂NH₂, —CHO, —COR′″, and —CN, R′ being selected from H and C₁-C₃ straight- and branched-chain alkyl. R″ from C₁-C₄ straight- and branched-chain alkyl and R′″ from C₁-C₃ straight- and branched-chain alkyl:

Y is selected from H, —COOH and —OR′″, where R′″ has the significance previously referred to; and Z is H; with the provisos that
(iii) When Y is H and X is any of the abovementioned significances, except —CN and —COOH, X is in the 4-position;
(iv) when Y has a significance other than H, it is in the 5-position when X is in the 2-position and in the 6-position when X is in the 3-position.

The compounds give a cooling sensation that can be more powerful than the best current commercial materials.

### CROSS REFERENCE TO RELATED APPLICATIONS

This application is a continuation-in-part of U.S. Ser. No. 11/437,294, filed May 19, 2006 which is a continuation-in-part of International Application No. PCT/CH2004/000646 filed Oct. 28, 2004., under 35 USC §120 and §365(c), which claims the benefit of the filing date of U.S. Provisional Application No. 60/523,977 filed Nov. 21, 2003.

This invention relates to cooling compounds.

Cooling compounds, that is, chemical compounds that impart a cooling sensation to the skin or the mucous membranes of the body, are well known to the art and are widely used in a variety of products such as foodstuffs, tobacco products, beverages, dentifrices, mouthwashes and toiletries.

One class of cooling compounds that have enjoyed substantial success consists of N-substituted p-menthane carboxamides. Examples of these compounds are described in, for example, British Patents GB 1.351.761-2 and U.S. Pat. No. 4,150,052.

It has now been found that a particular selection of such compounds exhibits a cooling effect that is both surprisingly strong and long-lasting. There is therefore provided a compound of the formula I

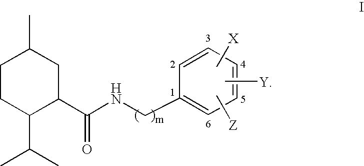

in which m is 0 or 1, Y and Z are selected independently from the group consisting of H, OH, C1-C4 straight or branched alklyl, or, a C1-C4 straight or branched alkoxy, X is (CH₂)ₙ—R, where n is 0 or 1 and R is a group selected from halogens, OH, OMe, NO₂, CN, Ac, SO₂NH₂, CHO, CO₂H and C₁-C₄ alkyl carboxylates, with the provisos that:

(a) when Y and Z are H, X is not F, OH, MeO or NO₂ in the 4-position and is not OH in the 2 or 6-position,

(b) when Y or Z is H then X, Y and Z are such that
(i) the groups in the 3- and 4-positions are not both OMe,
(ii) the groups in the 4- and 5-positions are not both OMe,
(iii) the groups in 3- and 5-positions are not OMe if the group in the 4-position is OH, and
(iv) the groups in the 3- and 5-positions are not OH if the group in the 4-position is methyl.

Particular subject cooling compounds include those in which X is in the 4-position. The most preferred compounds are When X is in the 4-position and Y and Z are H, OH, Me or OMe.

The cooling compounds of formula I have 3 chiral centres, giving rise to 8 stereoisomers. All possible stereoisomers are included in the scope of the present embodiments. The resolution of stereoisomers is well within the skill of the art, but it can add to the cost of these compounds, sometimes substantially. It may therefore often be preferred to use the compounds as racemic mixtures simply for economic reasons. However, if it is desired to prepare individual stereoisomers, this may be achieved according to methodology known in the art, e.g. preparative HPLC and GC on chiral stationary phases, by stereoselective synthesis, or starting from available chiral raw materials.

In a particular embodiment, the configuration of the compound is (1R, 2S, 5R).

In particular embodiments of Formula I;

(A) In is 0 or 1; X is —(CH₂)ₙ—R, where n is 0 or 1 and R is selected from the group consisting of —OR′, —COOH, —COOR″, —SO₂NH₂, —CHO, —COR′″, and —CN, R′ being selected from H and C₁-C₃ straight- and branched-chain alkyl R″ being selected from C₁-C₄ straight- and branched-chain alkyl and R′″ being selected from C₁-C₃ straight- and branched-chain alkyl; and Y is selected from the group consisting of H, —COOH and —OR′″, where R′″ has the significance previously referred to; and Z is H; with the provisos that
(i) when Y is H, X is not —OH, —CN, —OMe or —COOH in the 4-position and not —OH in the 2-position;
(ii) when Y has a significance other than H, Y is in the 5-position When X is in the 2-position and in the 6-position when X is in the 3-position;
(iii) the groups in the 3- and 4-positions are not both OMe,
(iv) the groups in the 4- and 5-positions are not both OMe,
(v) the groups in 3- and 5-positions are not OMe if the group in the 4-position is OH, and
(vi) the groups in the 3- and 5-positions are not OH if the group in the 4-position is methyl.

(B) m, X and Y are as in embodiment (A),A with the provisos that
(i) when Y is H and X is any of the abovementioned significances, except —CN and —COOH, X is in the 4-position;
(ii) when Y is a moiety other than H, Y is in the 5-position when X is in the 2-position and in the 6-position when X is in the 3-position.

(C) (a) m=0.
(b) X is in either the 2- or the 4-position, and when in the 2-position is CN and when in the 4-position is selected from the group consisting of —COOH, —COOCH₃, —COOCH₂CH₃, —CN and —SO₂NH₂; and
(c) Y is selected from —CH₃ in the 2-position and —OCH₃ in the 3-position.

Other particular embodiments are cooling compounds of the following formulae set forth in Table A (the accompanying numbers are the numbers of the relevant examples).

TABLE A

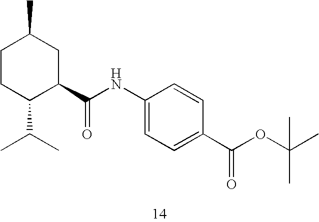

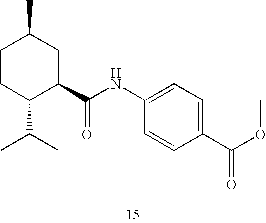

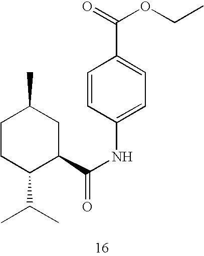

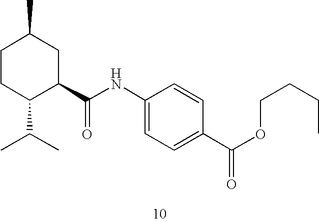

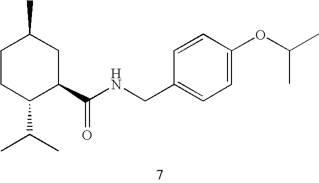

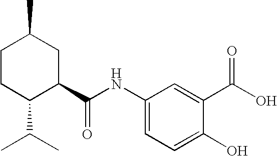

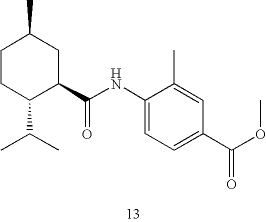

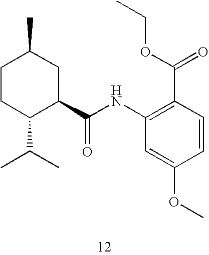

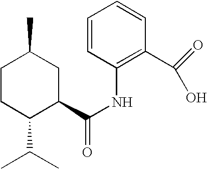

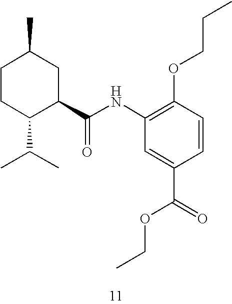

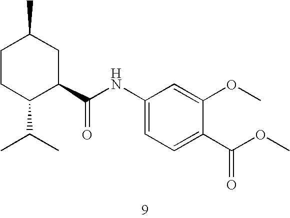

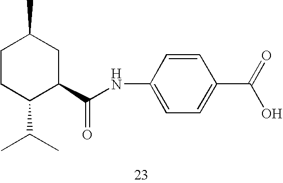

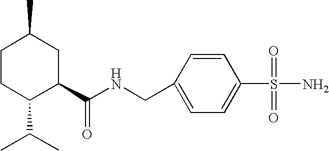

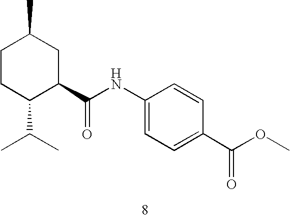

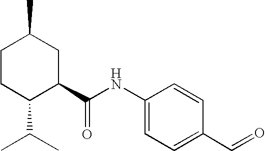

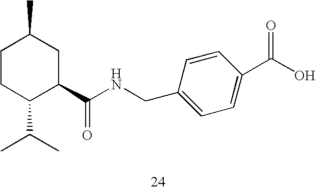

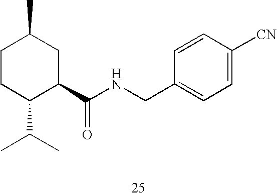

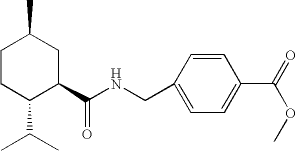

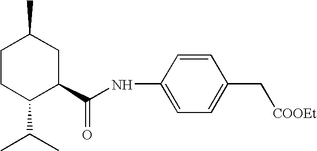

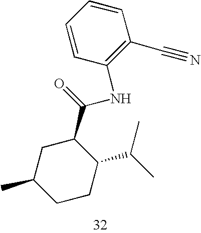

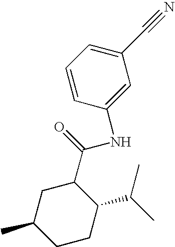

In other particular embodiments, the 2-isopropyl-5-methylcyclohexane moiety has the stereochemical configuration (1R, 2S, 5R), and

(a) m=0.

(b) X is in either the 2- or the 4-position, and ashen in the 2-position is CN and When in the 4-position is selected from the group consisting of —COOH, —COOCH₃, —COOCH₂CH₃, —CN and —SO₂NH₂; and

(c) Y is selected from —CH₃ in the 2-position and —OCH₃ in the 3-position.

The present cooling compounds may be prepared by reaction of arylalkylamine derivative with an appropriate acid chloride or carbonyl chloride. The carbonyl chloride may be prepared from 1-menthol ((1R, 2S, 5R)-2-isopropyl-5-methyl-cyclohexanol).

They are distinguished from similar compounds of the prior art by their surprisingly high cooling effect (up to 10 times higher than that of similar known compounds) and by the longevity of the cooling effect, which adds to their attractiveness in a large variety of products.

For example, a small group of panelists was asked to taste various solutions of cooling compounds and indicate which solutions had a cooling intensity similar or slightly higher than that of a solution of menthol at 2 ppm. In a second experiment, the same panel was asked to taste the solutions at the chosen concentrations and to record the cooling intensity at regular time intervals until no cooling could be sensed in the mouth. Results are shown in table 1.

TABLE 1
experiment on cooling intensity and longevity.

| | Concen- | |
|---|---|---|
| Chemical | tration | Longevity |
| 1-Menthol | 2.0 ppm | 35 minutes |
| N-ethyl p-menthanecarboxamide (WS-3) | 1.5 ppm | 57 minutes |
| Formula 1, m = 0, Y = Z = H, X = 4-CN | 0.5 ppm | 90 minutes |
| Formula 1, m = 0, Y = Z = H, X = 4-CH₂CN | 0.2 ppm | 93 minutes |

From Table 1, it can be seen that the compounds of Formula I are up to 10 times stronger and last up to 3 times longer than menthol, the reference cooling compound. Compounds of Formula I are also much stronger and last longer than WS-3, the best cooling compound of the prior art.

The subject cooling compounds may be used in products that are applied to the mouth or the skin to give a cooling sensation. By “applying” is meant any form of bringing into contact, for example, oral ingestion or, in the case of tobacco products, inhalation. In the case of application to the skin, it may be, for example, by including the compound in a cream or salve, or in a sprayable composition. Therefore, also provided is a method of providing a cooling effect to the mouth or skin by applying thereto a product comprising a compound as hereinabove described. Specific examples of products include, but are not limited to, the following:

Consumable products, including, but not limited to all food products, food additives, nutraceuticals, pharmaceuticals and any product placed in the mouth including chewing gum, oral care products, and oral hygiene products including but not limited to, cereal products: rice products, tapioca products, sago products, baker's products, biscuit products, pastry products, bread products, confectionery products, dessert products, gums, cheering gums, mouthwash, denial floss, flavored or flavor-coated straws, flavor or flavor-coated food/beverage containers; chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, yoghurts; cheese products, butter and butter substitute products, milk substitute products, soy products, edible oils and fat products, medicaments, beverages, carbonated beverages, alcoholic drinks such as beers, wines and spirits, non-alcoholic drinks such as soft drinks, mineral and aerated waters, fruit drinks, fruit juices, coffee, artificial coffee, tea, cocoa, including forms requiring reconstitution including, without limitation, beverage powder, milk based beverage powder, sugar-free beverage powder, beverage syrup, beverage concentrate, instant coffee, instant tea. instant cocoa, and coffee whitener food extracts, plant extracts, meat extracts, condiments, gelatins, pharmaceutical and non-pharmaceutical gums, tablets, lozenges, drops, emulsions, elixirs, syrups and other preparations for making beverages, and combinations thereof.

Oral care products, as hereinabove mentioned, include any composition applied to the oral cavity for the purposes of cleaning, freshening, healing, deodorising the cavity or any part thereof, may include, but are not limited to, toothpastes, tooth gels, tooth powders, tooth whitening products, mouthwashes, lozenges, dental floss, toothpicks, anti-plaque and anti-gingivitis compositions, throat lozenges, throat drops, inflammatory compositions, compositions for treatment of nasal symptoms, cold symptoms and upper gastrointestinal tract distress, compositions for cold relief, for alleviating discomfort of hot flash: gargle compositions.

Cosmetic products, such as aftershave lotions, baby products, including lotions, oils, powders, creams and shampoos, basecoats and undercoats, bath preparations- including capsules, oils, tablets, salts, soaps and detergents, beard softeners, blushers, body and hand preparations. bubble baits, cleaning products, colognes and toilet waters, cuticle softeners, dentifrices. deodorants, depilatories, douches, eye lotions, eye makeup preparations including eye makeup removers, else shadows, eyebrow pencils and eyeliners, face and neck preparations, face powders, feminine hygiene deodorants, foot powders and sprays, foundations, fragrance preparations, hair and scalp preparations including bleaches, colour spray s and other colouring preparations such as dyes and colours, hair lighteners with colour, hair conditioners, hair preparations, hair rinses, hair shampoos, hair sprays, hair straighteners, hair tints, hair tonics. hair wave sets, indoor tanning preparations, leg and body paints, lipsticks, makeup bases, makeup preparations including fixatives, manicuring preparations, mascara, men's talcum, moisturising preparations, nail creams and lotions, nail extenders, nail polish and enamel removers, nail polish and enamels, night skin care preparations, paste masks, perfumes, permanent waves, personal cleanliness products, powders, preshave lotions, rouges, sachets, shampoos, shaving creams shaving preparations miscellaneous, shaving soap, skin care preparations, including fresheners, suntan preparations including gels, creams and liquids.

The subject cooling compounds may be used alone or in combination with other cooling compounds known in the art, e.g., menthol, menthone, isopulegol, N-ethyl p-menthanecarboxamide (WS-3), N,2,3-trimethyl-2-isopropylbutanamide (WS-23), menthyl lactate (Frescolat™ ML), menthone glycerine acetal (Frescolat™ MGA), mono-menthyl succinate (Physcool™), mono-menthyl glutarate, O-menthyl glycerine (CoolAct™ 10), menthyl-N,N-dimethylsuccinamate and 2-sec-butylcyclohexanone (Freskomenthe™).

The subject cooling compounds are nov further described by, means of the following non-limiting examples, which describe particular embodiments.

The starting compound p-menthane-3-carbonyl chloride as used for the preparation of the compounds in Example 1-31 was prepared from 1-menthol.

### EXAMPLE 1

### Preparation of N-(4-cyanomethylphenyl) p-menthanecarboxamide

To a flask were added 6.6 g (50 mmol) of 4-aminobenzyl cyanide, 4.04 mL of pyridine and 100 mL MtBE. To this mixture, 10 b of p-menthane-3-carbonxyl chloride were added dropwise over 5 minutes. The reaction mixture was stirred for 24 h. To the reaction mixture, 50 mL of water were added. The mixture as separated. The organic layer was washed with 50 mL of water and 50 mL of brine. The organic laver was dried over MgSO₄. The solvent as evaporated in vacuo to afford the crude product, which w as recrystallized from hexanes to afford 10.1 g of the desired product with the following spectroscopic properties:

MS: 29.9 ([M+1]), 298 ([M³⁰]) ,132,83

¹H NMR (300 MHz; CDCl₃) δ: 7.58 (d 2H), 7.49 (s, 1H), 7.27 (d, 2H): 3.73 (s, 2H), 2.2 (t, 1H), 1.96-1.57 (m, 5H), 1.48-1.21 (m, 2H), 1.172- 0.99 (m, 2H), 0.94 (d, 3H), 0.93 (d, 3H), 0.85 (d, 3H)

¹³C NMR (75 MHz. CDCl₃)δ: 174.4, 137.8, 128.3, 125.1, 120.3, 118.2, 50.5, 44.3, 39.25, 34.3, 32.1, 28.7, 23.8, 22.9, 22.1, 21.2, 16.1

### EXAMPLE 2

### Preparation of N-(4-sulfamoylphenyl) p-menthanecarboxamide

A preparation similar to that described in example 1 gives the desired product with the following spectroscopic properties:

MS: 339([M+1]), 338([M⁺]), 172, 83

¹H NMR (300 MHz; DMSO) δ: 10.21 (s, 1H), 7.76 (d, 1H) 7.73 (d, 2H), 7.23 (s, 2H), 2.26-2.42 (m, 1H), 1.45-1.85 (m, 5H), 1.29-1.44 (m, 2H), 0.89 (d, 3H), 0.86 (d,3H), 0.78 (d, 3H)

¹³C NMR (75 MHz; DMSO) δ: 174.6, 142.3, 138.3, 126.7, 118.8, 48.9, 43:7, 34.3, 31.9, 28.6, 23.7, 22.35, 21.3, 16.25

### EXAMPLE 3

### Preparation of N-(4-cyanophenyl) p-menthanecarboxamide:

A preparation similar to that described in example 1 gives the desired product with the following spectroscopic properties:

MS: 285([M+1]), 284 ([M⁺]), 139.83

¹H NMR (300 MHz; CDCl₃) δ: 7.69 (d, 2H), 7.6 (d, 2H), 7.5 (s, 1H), 1.85-1.97 (m, 1H), 1.69-1.84 (m, 3H), 1.55-1.69 (m, 2H), 1.21-1.47 (m, 2H), 0.979-1.16 (m, 2H), 0.95 (d, 3H), 0.93 (d, 3H), 0.82 (d, 3H)

¹³C NMR (300 MHz; CDCl₃) δ: 174.6, 133.1, 119.4, 118.7, 100.35, 50.7, 44.4, 39.25, 34.2, 32.1, 2.8, 23.7, 22.0, 21.2,: 16.1, 14.0

### EXAMPLE 4

### Preparation of N-(4-acetylphenyl) p-menthanecarboxamide

A preparation similar to that described in example 1 gives the desired product with the following spectroscopic properties:

MS: 302([M+1]), 301([M⁺]), 135, 83

¹H NMR (300 MHz; CDCl₃) δ: 7.93 (d, 2H) 7.66 (d, 2H), 7.63 (s, 1H ), 2.57 (s, 3H), 2.09-2.31 (m, 1H), 1.84-1.98 (m, 1H), 1.68-1.85 (m, 5H), 1.56-1.68 (m, 2H), 1.17-1.48 (m, 2H),), 0.93 (d, 3H), 0.91 (d, 3H), 0.83 (d, 3H)

¹³C NMR (75 MHz; CDCl₃) δ: 197.1, 174.9, 142.7, 129.9, 119.2, 51.2, 44.9, 39.8, 34.8, 32.6, 29.2, 26.6, 24.3, 22.4, 21.5, 16.6

### EXAMPLE 5

### Preparation of N-(4-hydroxymethylphenyl) p-menthanecarboxamide

A preparation similar to that described in example 1 gives the desired product with the following spectroscopic properties:

MS: 290 (M+1), 289 (M⁺), 123, 83

¹HNMR (300 MHz, DMSO) δ: 9.9 (s, 1H) 7.54 (d, 2H), 7.21 (d, 2H), 4.2 (s, 2H), 2.36-2.1 (m, 1H), 1.8-1.59 (m, 6H), 1.57-1.44 (m, 1H), 1.21-0.9 (m, 4H), 0.87 (dd, 3H), 0.85 (dd, 3H) 0.79 (d, 2H)

¹³C NMR (75 MHz; DMSO) δ: 173.7, 137.7, 137.1, 126.7, 118.9, 62.6, 48.6, 43.6. 34.2, 31.7, 28.3, 23.6, 22.2, 21.1, 16.1

### EXAMPLE 6

### Preparation of N-(3-hydroxy-4-methoxyphenyl) p-menthanecarboxamide

A preparation similar to thai described in example I gives the desired product with the following spectroscopic properties:

MS: 306([M+1]). 305([M⁺]), 139, 83

¹H NMR (300 MHz; CDCl₃) δ: 7.14 (s, 1H), 7.08 (d, 1H), 6.78 (d, 1H), 5.7 (s, 1H), 3.8 (s, 3H), 2.02-2.21 (m, 2H), 1.53-1.94 (m, 5H), 1.17-1.48 (m, 2H), 0.97-1.17 (m, 2H), 0.92 (dd, 3H), 0.91 (dd, 3H), 0.82 (d, 3H)

¹³C NMR (75 MHz, CDCl₃) δ: 173.9, 145.6, 143.3, 131.7. 111.65, 110.8, 107.4, 56.1. 50.5, 44.4, 39.2 32.15. 34.4. 28.6 23.8, 22.1, 21.2 16.1

### EXAMPLES 7-31

The following compounds were made by methods analogous to those previously described.

TABLE B

| Example No. | Compound and properties |
|---|---|
| 7 | (1R,2S,5R)-N-(4-isopropoxybenzyl)-2-isopropyl-5- |
| | methylcyclohexanecarboxamide |
| | ¹H NMR (DMSO) δ: 8.36-8.15 (t, 1H), 7.20-7.02 (d, 2H), 6.91-6.76 |
| | (d, 2H), 4.70-4.42 (m, 1H), 4.28-4.05 (d, 2H), 2.19-2.07 (t, 1H), |
| | 1.79-1.30 (m, 6H), 1.28-1.20 (d, 6H), 1.15-1.05 (m, 1H), 0.92-0.70 |
| | (m, 11H). LC-MS: 332.2 (M⁺), 354.3 (M⁺²²). |
| 8 | (1R,2S,5R)-2-isopropyl-5-methyl-N-(4-propionylphenyl)- |
| | cyclohexanecarboxamide |
| | ¹H NMR (CDCl₃) δ: 8.01-7.87 (d, 2H), 7.70-7.57 (d, 2H), 3.05-2.88 |
| | (q, 2H), 2.25-2.10 (t, 1H), 1.99-1.87 (d, 1H), 1.85-1.58 (m, 4H), |
| | 1.52-1.15 (m, 5H), 1.12-0.96 (m, 2H), 0.94-0.89 (d, 6H), 0.87-0.78 |
| | (d, 3H). LC-MS: 338.0 (M⁺²³). |
| 9 | methyl 4-((1R,2S,5R)-2-isopropyl-5-methylcyclohexane- |
| | carboxamido)-2-methoxybenzoate |
| | ¹H NMR (CDCl₃) δ: 7.89-7.67 (m, 2H), 6.83-6.68 (d, 1H), 4.00- |
| | 3.91 (s, 3H), 3.90-3.81 (s, 3H), 2.25-2.03 (t, 1H), 1.97-1.50 (m, 7H), |
| | 1.47-1.21 (m, 2H), 1.15-0.89 (m, 8H), 0.87-0.78 (d, 3H). |
| | LC-MS: 346.0 (M⁻) |
| 10 | butyl 4-((1R,2S,5R)-2-isopropyl-5-methylcyclohexane- |
| | carboxamido)benzoate |
| | ¹H NMR (DMSO) δ: 10.32-10.15 (s, 1H), 7.94-7.82 (d, 2H), 7.80- |
| | 7.69 (d, 2H), 4.31-4.16 (t, 2H), 2.42-2.27 (t, 1H), 1.87-1.30 (m, |
| | 10H), 1.21-1.09 (m, 1H), 0.99-0.72 (m, 14H). LC-MS: 358.2 (M⁻). |
| 11 | ethyl 3-((1R,2R,5R)-2-isopropyl-5-methylcyclohexane- |
| | carboxamido)-4-propoxybenzoate |
| | ¹H NMR (CDCl₃) δ: 9.01 (s, 1H), 7.87-7.70 (m, 2H), 6.93-6.82 (d, |
| | 1H), 4.42-4.26 (q, 2H), 4.12-3.99 (t, 2H), 2.27-2.11 (td, 1H), 1.98- |
| | 1.67 (m, 6H), 1.65-1.59 (m, 1H), 1.45-1.27 (t, 5H), 1.12-0.97 (t, |
| | 5H), 0.97-0.89 (m, 6H), 0.85-0.79 (d, 3H). LC-MS: 412.3 (M⁺²³). |
| 12 | ethyl 2-((1R,2S,5R)-2-isopropyl-5-methylcyclohexane- |
| | carboxamido)-4-methoxybenzoate |
| | ¹H NMR (CDCl₃) δ: 11.40 (s, 1H), 8.50 (s, 1H), 8.02-7.88 (d, 1H), |
| | 6.65-6.52 (d, 1H), 4.45-4.28 (q, 2H), 3.87 (s, 3H), 2.36-2.21 (td, |
| | 1H), 1.98-1.60 (m, 5H), 1.47-1.22 (m, 5H), 1.12-0.97 (m, 2H), 0.96- |
| | 0.89 (d, 6H), 0.88-0.80 (d, 3H). LC-MS: 384.3 (M⁺²³). |
| 13 | methyl 4-((1R,2S,5R)-2-isopropyl-5-methylcyclohexane- |
| | carboxamido)-3-methylbenzoate |
| | ¹H NMR (CDCl₃) δ: 8.23-8.12 (d, 1H), 7.95-7.82 (m, 2H), 7.11- |
| | 6.99 (s, 1H), 3.91 (s,3H), 2.31 (s, 3H), 2.28-2.15 (t, 1H), 1.99-1.89 |
| | (d, 1H), 1.88-1.68 (m, 3H), 1.66-1.60 (m, 1H), 1.48-1.22 (m, 2H), |
| | 1.18-0.97 (m, 2H), 0.96-0.90 (d, 6H), 0.88-0.81 (d, 3H). LC-MS: |
| | 332.1 (M⁺), 354.1. (M⁺²³). |
| | MP: 147-149° C. |
| 14 | tert-butyl 4-((1R,2S,5R)-2-isopropyl-5-methylcyclohexane- |
| | carboxamido)benzoate |
| | MS: 359, 303, 386, 260, 248, 235, 221, 207, 193, 179, 167, 137, |
| | 136, 120, 97, 83, 69, 55, 41, 32 |
| 15 | methyl 4-((1R,2S,5R)-2-isopropyl-5-methylcyclohexane- |
| | carboxamido)benzoate |
| | MS: 317, 302, 286, 260, 248, 221, 206, 193, 178, 167, 151, 139, |
| | 135, 120, 97, 83, 69, 57, 55, 41 |
| | Mp: 136-138° C. |
| 16 | ethyl 4-((1R,2S,5R)-2-isopropyl-5-methylcyclohexane- |
| | carboxamido)benzoate |
| | ¹H NMR (CDCl₃) δ: 8.05-7.95 (d, 2H), 7.67-7.59 (d, 2H), 7.46-7.40 |
| | (s, 1H), 4.44-4.29 (t, 2H), 2.25-2.1 (td, 1H), 1.97-1.86 (d, 1H), 1.85- |
| | 1.58 (m, 4H), 1.45-1.35 (m, 4H), 1.35-1.22 (t, 2H), 1.11-0.98 (m, |
| | 1H), 0.97-0.88 (q, 6H), 0.87-0.77 (d, 3H). |
| | ¹³C NMR (CDCl₃) δ: 224, 182.2, 174.4, 141.95, 130.6, 118.6, 60.7, |
| | 50.8, 44.4, 39.3, 34.3, 32.1, 28.7, 23.8, 22.1, 21.2, 16.1, 14.2. |
| | GC-MS: 331 (M), 286, 220, 207, 165, 139, 120, 97, 83, 55, 41. |
| | MP: 167-169 C. |
| 17 | 2-[(2-Isopropyl-5-methyl-cyclolhexanecarbonyl)-amino]-benzoic |
| | acid methyl ester |
| | ¹H NMR (CDCl₃) δ: 11.21-11.05 (s, 1H), 8.88-8.72 (d, 1H), 8.11- |
| | 7.97 (d, 1H), 7.65-7.47 (t, 1H), 7.15-6.99 (t, 1H), 3.99-3.88 (s, 3H), |
| | 2.37-2.20 (td, 1H), 2.03-1.88 (d, 1H), 1.87-1.57 (m, 4H), 1.54-1.22 |
| | (m, 3H), 1.20-0.99 (m, 2H), 0.98-0.88 (dd, 6H), 0.88-0.79 (d, 3H). |
| | ¹³C NMR (CDCl₃) δ: 175.1, 168.6, 141.6, 134.5, 130.6, 122.06, |
| | 120.2, 114.6, 100.3, 52.1, 51.6, 44.5, 39.3, 34.4, 32.1, 28.7, 23.8, |
| | 22.1, 21.2, 15.9. |
| | GC-MS: 317 (M), 286, 221, 193, 174, 151, 146, 119, 83, 55, 41. |
| 18 | 2-((1R,2S,5R)-2-isopropyl-5-methylcyclohexane- |
| | carboxamido)benzoic acid |
| | ¹H NMR (CDCl₃) δ: 11.1-10.97 (s, 1H), 8.88-8.76 (d, 1H), 8.23-8.1 |
| | (d, 1H), 7.7-7.52 (t, 1H), 7.21-7.06 (t, 1H), 2.42-2.22 (td, 1H), 2.06- |
| | 1.9 (d, 1H), 1.9-1.55 (m, 5H), 1.54-1.22 (m, 4H), 1.21-1.01 (m, 2H), |
| | 1.0-0.91 (q, 6H), 0.89-0.79 (d, 3H). |
| | ¹³C NMR (CDCl₃) δ: 175.6, 172.2, 141.9, 135.5, 131.6, 122.5, |
| | 120.5, 113.8, 51.7, 44.6, 39.3, 34.4, 32.1, 31.4, 28.7, 23.8, 22.5, |
| | 22.1, 21.2, 15.9. |
| | GC-MS: 303 (M), 260, 221, 207, 179, 146, 137, 119, 95, 83, 55, 41. |
| 19 | (1R,2S,5R)-N-(4-formylphenyl)-2-isopropyl-5-methylcyclo- |
| | hexanecarboxamide |
| | GC-MS: 287 (M), 244, 191, 176, 167, 139, 121, 83, 69, 55, 41. |
| 20 | 2-Isopropyl-5-methyl-cyclohexanecarboxylic acid (2,4- |
| | dimethoxy-phenyl)-amide |
| | ¹H NMR (CDCl₃) δ: 8.32-8.23 (d, 1H), 7.59-7.49 (s, 1H), 6.52-6.43 |
| | (2 s, 2H), 3.89-3.83 (s, 3H), 3.81-3.75 (s, 3H), 2.23-2.1 (td, 1H), |
| | 1.96-1.56 (m, 5H), 1.49-1.24 (m, 2H), 1.17-0.97 (m, 2H), 0.96-0.88 |
| | (d, 6H), 0.87-0.77 (d, 3H). |
| | ¹³C NMR (CDCl₃) δ: 173.6. 156.0, 148.9, 121.2, 120.5. 103.5, |
| | 100.3, 98.4, 55.5, 55.4, 50.7, 44.5, 39.3, 34.4, 32.1, 28.5, 23.8, 22.1, |
| | 21.2, 15.9. |
| | GC-MS: 319 (M), 248, 223, 195, 179, 153, 138, 110, 95, 83, 55, 41. |
| 21 | 2-Isopropyl-5-methyl-cyclohexanecarboxylic acid (2-hydroxy-4- |
| | methyl-phenyl)-amide |
| | ¹H NMR (CDCl₃) δ: 8.42-8.32 (s, 1H), 7.96-7.88 (s, 1H), 7.25-7.18 |
| | (m, 1H), 7.04-6.95 (d, 1H), 6.46-6.36 (d, 1H), 2.25-2.19 (s, 3H), |
| | 2.18-2.08 (m, 1H), 1.96-1.58 (m, 6H), 1.45-1.25 (m, 2H), 1.17-0.95 |
| | (m, 2H), 0.95-0.91 (d, 3H), 0.90-0.86 (s, 3H), 0.85-0.77 (d, 3H). |
| | ¹³C NMR (CDCl₃) δ: 175.1, 155.5, 136.1, 130.2, 120.8, 109.8, |
| | 107.3, 50.9, 44.2, 39.0, 34.3, 32.2, 28.6, 23.9, 22.1, 21.0, 16.1, 15.4. |
| | GC-MS: 289 (M), 246, 178, 165, 123, 83, 55, 41. |
| 22 | ethyl 2-((1R,2S,5R)-2-isopropyl-5- |
| | methylcyclohexanecarboxamido)benzoate |
| | ¹H NMR (CDCl₃) δ: 11.3-11.02 (s, 1H), 8.87-8.71 (d, 1H), 8.11- |
| | 7.96 (d, 1H), 7.61-7.43 (t, 1H), 7.12-6.97 (t, 1H), 4.50-4.29 (q, 2H), |
| | 2.38-2.17 (td, 1H), 2.01-1.87 (d, 1H), 1.86-1.57 (m, 4H), 1.52-1.40 |
| | (t, 3H), 1.38-1.21 (m, 2H), 1.15-0.98 (m, 2H), 0.96-0.88 (dd, 6H), |
| | 0.85-0.76 (d, 3H). |
| | ¹³C NMR (CDCl₃) δ: 175.2, 168.3, 141.8, 134.5, 130.7, 122.1, |
| | 120.4, 115.1, 61.3, 51.8, 44.7, 39.5, 34.6, 32.3, 28.9, 24.0, 22.3, |
| | 21.3, 16.1, 14.2. |
| | GC-MS: 331 (M), 286, 220, 207, 174, 165, 146, 119, 95, 83, 69, 55, |
| | 41. |
| 23 | 4-((1R,2S,5R)-2-isopropyl-5-methylcyclohexane- |
| | carboxamido)benzoic acid |
| | MP: 262-264 C. |
| | ¹H NMR (CDCl₃/MeOD) δ: 8.0 (d, 2H), 7.7 (d, 2H), 2.3 (dt, 1H), |
| | 1.9-1.6 (m, 5H), 1.4-1.2 (m, 2H), 1.15-1.0 (m, 2H), 0.94, d, 6H), |
| | 0.85 (d, 3H) |
| 24 | 4-(((1R,2S,5R)-2-isopropyl-5-methylcyclohexane- |
| | carboxamido)methyl)benzoic acid |
| | MP: 220-225 C. |
| 25 | (1R,2S,5R)-N-(4-cyanobenzyl)-2-isopropyl-5- |
| | methylcyclohexanecarboxamide |
| | MP: 148-154 C. |
| | ¹H NMR (CDCl₃/MeOD) δ: 7.62 (d, 2H), 7.37 (d, 2H), 5.89 (b, 1H), |
| | 4.5 (d, 2H), 2.07 (dt, 1H), 1.85-1.65 (m, 4H), 1.6-1.5 (m, 1H), 1.4- |
| | 1.2 (m, 2H), 1.15-0.95 (m, 2H), 0.9 (d, 6H), 0.78 (d, 3H) |
| 26 | 2-hydroxy-5-((1R,2S,5R)-2-isopropyl-5-methylcyclohexane- |
| | carboxamido)benzoic acid |
| 27 | (1R,2S,5R)-2-isopropyl-5-methyl-N-(4-sulfamoylbenzyl)- |
| | cyclohexanecarboxamide |
| | MP: 160-170 |
| | ¹H NMR (CDCl₃/MeOD) δ: 7.82 (t, 2H), 7.32 (m, 3H), 4.44 (d, 2H), |
| | 2.2-2.0 (m, 1H), 1.9-1.6 (m, 5H), 1.6-1.45 (m, 1H), 1.4-1.1 (m, 2H) |
| | 1.1-1.0 (m, 2H), 0.94-0.87 (m, 6H), 0.83-0.75 (m, 3H). |
| 28 | 4-(((1R,2S,5R)-2-isopropyl-5-methyl-cyclohexanecarboxamido)- |
| | methyl)benzoic acid |
| 29 | ethyl 2-(4-((1R,2S,5R)-2-isopropyl-5-methylcyclohexane- |
| | carboxamido)phenyl)acetate |
| 30 | (1R,2S,5R)-N-(2-cyanophenyl)-2-isopropyl-5- |
| | methylcyclohexane-carboxamide |
| | ¹H NMR (CDCl₃) δ: 8.46 (d, 1H), 7.62-7.56 (m, 3H), 7.14 (t, 1H), |
| | 2.30-2.23 (td, 1H), 1.98-1.93 (d, 1H), 1.82-1.62 (m, 4H), 1.36-1.28 |
| | (m, 2H), 1.12-1.02 (m, 2H), 0.99-0.93 (dd, 6H), 0.87-0.85 (d, 3H). |
| | ¹³C NMR (CDCl₃) δ: 174.58, 140.59, 134.15, 132.07, 123.94, |
| | 132.07, 123.94, 121.29, 116.32, 101.81, 51.0, 44.84, 39.44, 34.43 |
| | 32.23, 28.99, 23.96, 22.21, 21.29, 16.23 |
| 31 | (2S,5R)-N-(3-cyanophenyl)-2-isopropyl-5-methylcyclohexane- |
| | carboxamide |
| | ¹H NMR (CDCl₃) δ: 8.15 (s, 1H), 7.98 (s, 1H), 7.80-7.75 (td, 1H), |
| | 7.43-7.33 (m, 2H), 2.30-2.23 (td, 1H), 1.92-1.85 (d, 1H), 1.82-1.55 |
| | (m, 4H), 1.40-1.20 (m, 2H), 1.10-0.95 (m, 2H), 0.95-0.87 (dd, 6H), |
| | 0.87-0.83 (d, 3H). |
| | ¹³C NMR (CDCl₃) δ: 175.25, 139.10, 129.80, 127.45, 124.27, |
| | 123.16, 118.59, 112.69, 50.49, 44.50, 39.42, 34.44, 32.26, 28.98, |
| | 23.95, 22.23, 21.34, 16.31 |

### EXAMPLE 32

### Testing of Compounds

A small group of panelists were asked to taste various aqueous solutions of compounds. The compounds were first dissolved in ethanol at 1% by weight and then dosed into water. The panelists were asked to indicate which solutions had a cooling intensity similar to or slightly higher than that of a solution of menthol at 2 ppm. The results are shown in Table 2.

TABLE 2

| | Concen- | |
|---|---|---|
| Compound/ | tration | |
| Example No. | [ppm] | Description |
| 7 | 0.01 | slight delay in onset, builds to similar |
| | | or slightly cooler than menthol, teeth |
| | | and gums |
| 9 | 0.005 | Similar to menthol, refreshing, slightly |
| | | bitter long lasting |
| 15 | 2 | slightly cooling, flat cooling |
| 16 | 2 | Cooling similar to menthol |
| 19 | 2 | cooling builds and lingers |
| 23 | 2 | slightly burning then cooling |
| 24 | 2 | little cooling, slightly refreshing |
| 25 | 2 | good icy cooling, slightly astringent |
| 27 | 2 | Similar to reference, builds up |
| 28 | 2 | strong cooling up front, peppery heat |
| 29 | 2 | icy, crisp cool on tongue |
| 30 | 2 | clean, slightly stronger than menthol |
| 31 | 1 | weak cooling, tingling, numbing on tongue, |
| 32 | 2 | mouthcoating, more cooling than menthol |

### EXAMPLE 34

### Application in Mouthwash

| | | |
|---|---|---|
| Alcohol 95% | 177 | mL |
| Sorbitol 70% | 250 | g |
| Compound of example 1 as | 50 | mL |
| a 1% solution in alcohol | | |
| Peppermint oil, Terpeneless | 0.300 | g |
| Methyl salicylate | 0.640 | g |
| Eucalyptol | 0.922 | g |
| Thymol | 0.639 | g |
| Benzoic acid | 1.500 | g |
| Pluronic ™ F127 | 5.000 | g |
| Sodium Saccharin | 0.600 | g |
| Sodium Citrate | 0.300 | g |
| Citric Acid | 0.100 | g |

| | |
|---|---|
| Water | q.s. 1 liter |

The ingredients are mixed, 30 mL of obtained solution is put in the mouth, swished around. gargled and spit out. An intense cooling is felt in every area of the mouth as well as the lips. The cooling perception lasts for several hours.

### EXAMPLE 35

### Application in Toothpaste

| | |
|---|---|
| Opaque toothgel | 97.000 g |
| Compound of example 2 | 2.500 g |
| as a 2% solution in PG | |
| Peppermint oil, Terpeneless | 0.500 g |

The materials are mixed in the toothgel, and a panelist's teeth are brushed using this toothgel. The mouth is rinsed with water and the water spit out. An intense cooling sensation is felt by the panelist in all areas of the mouth. The cooling perception lasts for several hours.

It will be understood that the embodiments described herein are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

1. A compound of formula I 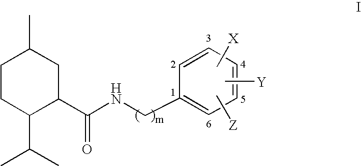
wherein m is 0 or 1;
X is —(CH₂)ₙ—R. Where n is 0 or 1 and R is selected from the group consisting of —OR′, —COOH, —COOR″, —SO₂NH₂, —CHO, —COR′″, and —CN, R″ being selected from the group consisting of H and C₁-C₃ straight- and branched-chain alkyl). R′″ being selected from the croup consisting of C₁-C₄ straight- and branched-chain alkyl and R′″ being selected from the group consisting of C₁-C₃ straight- and branched-chain alkyl; and
Y is selected from the group consisting of H, —COOH and —OR′″, where R′″ has the significance previously referred to; and Z is H;
with the provisos that
(i) when Y is H, X is not —OH, —CN, —OMe Or —COOH in the 4-position and not —OH in the 2-position;
(ii) when Y has a significance other than H, Y is in the 5-position when X is in the 2-position and in the 6-position when X is in the 3-position;
(iii) the groups in the 3- and 4-positions are not both OMe,
(iv) the groups in the 4- and 5-positions are not both OMe,
(v) the groups in 3- and 5-positions are not OMe if the group in the 4-position is OH, and;
(vi) the groups in the 3- and 5-positions are not OH if the croup in the 4-position is methyl.

2. A compound according to claim 1, in which X is in the 4-position.

3. A compound according to claim 2, in which Y is selected from the group consisting of H, OH, Me and OMe.

4. A compound according to claim 1, in which the 2-isopropyl-5-methylcyclohexane moiety has the stereochemical configuration (1R, 2S, 5R).

5. A compound according to claim 4, in which
(a) m=0
(b) X is in either the 2- or the 4-position, and when in the 2-position is CN and when in the 4-position is selected from the group consisting of —COOH, —COOCH₃, —COOCH₂CH₃, —CN and —SO₂NH₂; and
(c) Y is selected from —CH₃ in the 2-position and —OCH₃ in the 3-position.

6. A compound according to claim 1, in which.
(i) when Y is H and X is any of the significances of claim 1, except —CN and —COOH, X is in the 4-position;
(ii) when Y is a moiety other than H, Y is in the 5-position when X is in the 2-position, and Y is in the 6-position when X is in the 3-position.

7. A compound according to claim 1, in which
(a) m=0,
(b) X is in either the 2- or the 4-position, and when in the 2-position is CN and when in the 4-position is selected from the group consisting of —COOH, —COOCH₃, —COOCH₂CH₃, —CN and —SO₂NH₂; and
(c) Y is selected from —CH₃ in the 2-position and —OCH₃ in the 3-position.

8. A compound according to claim 1 selected from the group consisting of (1R, 2S, 5R)—N— (4-isopropoxybenzyl)-2-isopropyl-5-methylcyclohexanecarboxamide; (1R, 2S, 5R)-2-isopropyl-5-methyl-N-(4-propionylphenyl)-cyclohexanecarboxamide;
methyl 4-((1R, 2S, 5R)-2-isopropyl-5-methylcyclohexane-carboxamido)-2-methoxybenzoate; butyl 4-((1R, 2S, 5R)-2-isopropyl-5-methylcyclohexane-carboxamido)benzoate; ethyl 3-((1R, 2R, 5R)-2-isopropyl-5-methylcyclohexane-carboxamido)-4-propoxybenzoate; ethyl 2-((1R, 2S, 5R)-2-isopropyl-5-methylcyclohexane-carboxamido)-4-methoxybenzoate; methyl 4-((1R, 2S, 5R)-2-isopropyl-5-methylcyclohexane-carboxamido)-3-methylbenzoate; tert-butyl 4-((1R, 2S, 5R)-2-isopropyl-5-methylcyclohexane-carboxamido)benzoate; methyl 4-((1R, 2S, 5R)-2-isopropyl-5-methylcyclohexane-carboxamido)benzoate; ethyl 4-(1R, 2S, 5R)-2-isopropyl-5-methylcyclohexane-carboxamide)benzoate; 2-[(2-isopropyl-5-methyl-cyclohexanecarbonyl)-amino]-benzoic acid methyl ester; 2-((1R, 2S, 5R)-2-isopropyl-5-methylcyclohexane-carboxamido)benzoic acid; (1R, 2S, 5R)-N-(4-formylphenyl)-2-isopropyl-5-methylcyclo-hexanecarboxamide; 2-isopropyl-5-methyl-cyclohexanecarboxylic acid (2,4-dimethoxy-phenyl)-amide; 2-isopropyl-5-methyl-cyclohexanecarboxylic acid (2-hydroxy-4-methyl-phenyl)-amide; ethyl 2-((1R, 2S ,5R)-2-isopropyl-5-methylcyclohexanecarboxamido)benzoate; 4-((1R, 2S, 5R)-2-isopropyl-5-methylcyclohexane-carboxamido)benzoic acid; 4-(((1R, 2S, 5R)-2-isopropyl-5-methylcyclohexane-carboxamido)methyl)benzoic acid; (1R, 2S, 5R)-N-(4-cyanobenzyl)-2-isopropyl-5-methylcyclohexanecarboxamide; 2-hydroxy-5-((1R, 2S, 5R)-2-isopropyl-5-methylcyclohexane-carboxamido)benzoic acid; (1R, 2S, 5R)-2-isopropyl-5-methyl-N-(4-sulfamoylbenzyl)-cyclohexanecarboxamide; 4-(((1R, 2S, 5R)-2-isopropyl-5-methyl-cyclohexanecarboxamido)-methyl)benzoic acid; ethyl 2-(4-((1R, 2S, 5R)-2-isopropyl-5-methylcyclohexane-carboxamido)phenyl)acetate. (1R, 2S, 5R)-N-(2-cyanophenyl)-2-isopropyl-5--methylcyclohexane-carboxamide; and, (2S, 5R)-N-(3-cyanophenyl)-2-isopropyl-5-methylcyclohexane-carboxamide.

9. A method of providing a cooling effect to the mouth or skin by applying thereto a product comprising a compound according to claim 1.

10. The method of claim 9 wherein said applying, comprises inhalation.

11. The method of claim 9 wherein said applying comprises oral ingestion.

12. A method according to claim 9 wherein tile compound is selected from the group consisting of (1R, 2S, 5R)-N-(4-isopropoxybenzyl)-2-isopropyl-5-methylcyclohexanecarboxamide; (1R, 2S, 5R)-2-isopropyl-5-methyl-N-(4-propionylphenyl)-cyclohexanecarboxamide; methyl 4-((1R, 2S, 5R)-2-isopropyl-5-methylcyclohexane-carboxamido)-2-methoxybenzoate; butyl 4-((1R, 2S, 5R)-2-isopropyl-5-methylcyclohexane-carboxamido)benzoate; ethyl 3-((1R, 2R, 5R)-2-isopropyl-5-methylcyclohexane-carboxamido)-4-propoxybenzoate; ethyl 2-((1R, 2S, 5R)-2-isopropyl-5- methylcyclohexane-carboxamido)-4-methoxybenzoate; methyl 4-((1R, 2S, 5R)-2-isopropyl-5-methylcyclohexane-carboxamido)-3-methylbenzoate; tert-butyl 4-((1R, 2S, 5R)-2-isopropyl-5-methylcyclohexane-carboxamido)benzoate; methyl 4-((1R, 2S, 5R)-2-isopropyl-5-methylcyclohexane-carboxamido)benzoate; ethyl 4-((1R, 2S, 5R)-2-isopropyl-5-methylcyclohexane-carboxamido)benzoate; 2-[(2-isopropyl-5-methyl-cyclohexanecarbonyl)-amino]-benzoic acid methyl ester; 2-((1R, 2S, 5R)-2-isopropyl-5-methylcyclohexane-carboxamido)benzoic acid; (1R, 2S, 5R)-N-(4-formylphenyl)-2-isopropyl-5-methylcyclo-hexanecarboxamide; 2-isopropyl-5-methyl-cyclohexanecarboxylic acid (2-4-dimethoxy-phenyl)-amide; 2-isopropyl-5-methyl-cyclohexanecarboxylic acid (2-hydroxy-4-methyl-phenyl)-amide; ethyl 2-((1R, 2S, 5R)-2-isopropyl-5-methylcyclohexanecarboxamido)benzoate; 4-((1R, 2S, 5R)-2-isopropyl)5-methylcyclohexane-carboxamido)benzoic acid; 4-(((1R, 2S, 5R)-2- isopropyl-5-methylcyclohexane-carboxamido)methyl)benzoic acid; (1R, 2S, 5R)-N-(4-cyanobenzyl)-2-isopropyl-5-methylcyclohexanecarboxamide; 2-hydroxy-5-((1R, 2S, 5R)-2-isopropyl-5-methylcyclohexane-carboxamido)benzoic acid; (1R, 2S, 5R)-2-isopropyl-5-methyl-N-(4-sulfamoylbenzyl)-cyclohexanecarboxamide; 4-(((1R, 2)S, 5R)-2-isopropyl-5-methyl-cyclohexanecarboxamido)-methyl)benzoic acid- ethyl 2-(4-((1R, 2S, 5R)-2-isopropyl-5-methylcyclohexane-carboxamido)phenyl)acetate; (1R, 2S, 5R)-N-(2-cyanophenyl)-2-isopropyl-5-methylcyclohexane-carboxamide; and, (2S, 5R)-N-(3-cyanophenyl)-2-isopropyl-5-methylcyclohexane-carboxamide.

13. A product that is applied to the mouth or the skin comprising an effective amount of a compound according to claim 1, wherein the amount is effective to give a cooling sensation.

14. The product of claim 13 comprising at least one of tobacco products, consumable products, oral care products, or cosmetic products.

15. The product of claim 13 further comprising an additional cooling compound.

16. The product of claim 15 wherein the additional cooling compound comprises at least one of menthol, menthone, isopulegol, N-ethyl p-menthanecarboxamide, N,2,3-trimethyl-2-isopropylbutanamide, methyl lactate, menthone glycerine acetal, mono-menthyl succinate, mono-menthyl glutarate, O-menthyl glycerine, menthyl-N,N-dimethylsuccinamate, or 2-sec-butylcyclohexanone.

17. The product of claim 13 wherein the compound according to claim 1 is selected from the group consisting of (1R, 2S, 5R)-N-(4-isopropoxybenzyl)-2-isopropyl-5-methylcyclohexanecarboxamide; (1R, 2S, 5R)-2-isopropyl-5-methyl-N-(4-propionylphenyl)-cyclohexanecarboxamide; methyl 4-((1R, 2S, 5R)-2-isopropyl-5-methylcyclohexane-carboxamido)-2-methoxybenzoate butyl-4-((1R, 2S, 5R)-2-isopropyl-5-methylcyclohexane-carboxamido)benzoate; ethyl 3-((1R, 2R, 5R)-2-isopropyl-5-methylcyclohexane-carboxamido)-4-propoxybenzoate; ethyl 2-((1R, 2S, 5R)-2-isopropyl-5-methylcyclohexane-carboxamido)-4-methoxybenzoate: methyl) 4-((1R, 2S, 5R)-2-isopropyl-5-methylcyclohexane-carboxamido)-3-methylbenzoate; tert-butyl 4-((1R, 2S, 5R)-2-isopropyl-5-methylcyclohexane-carboxamido)benzoate; methyl 4-((1R, 2S, 5R)-2-isopropyl-5-methylcyclohexane-carboxamido)benzoate; methyl 4-((1R, 2S, 5R)-2-isopropyl-5-methylcyclohexane-carboxamido)benzoate; 2-[(2-isopropyl-5-methyl-cyclohexanecarbonyl)-amino]-benzoic acid methyl ester; 2-((1R, 2S, 5R)-2-isopropyl-5-methylcyclohexane-carboxamido)benzoic acid; (1R, 2S, 5R)-N-(4-formylphenyl)-2-isopropyl-5-methyl-cyclohexanecarboxylic acid (2,4-dimethoxy-phenyl)-amide; 2-isopropyl-5-methyl-cyclohexanecarboxylic acid (2-hydroxy-4-methyl-phenyl)-amide; ethyl 2-((1R, 2S, 5R)-2-isopropyl-5-methylcyclohexanecarboxamido)benzoate; 4-((1R, 2S, 5R)-2-isopropyl-5-methylcyclohexane-carboxamido)benzoic acid; 4-(((1R, 2S, 5R)-2-isopropyl-5-methylcyclohexane-carboxamido)methyl)benzoic acid-(1R, 2S, 5R)-N-(4-cyanobenzyl)-2-isopropyl-5-methylcyclohexanecarboxamide; 2-hydroxy-5-((1R, 2S, 5R)-2-isopropyl-5-methylcyclohexane-carboxamido)benzoic acid; (1R, 2S, 5R)-2-isopropyl-5-methyl)-N-(4-sulfamoylbenzyl)-cyclohexanecarboxamide; 4-(((1R, 2S, 5R)-2-isopropyl)-5-methyl-cyclohexanecarboxamido)-methyl)benzoic acid; methyl 2-(4-((1R, 2S, 5R)-2-isopropyl-5-methylcyclohexane-carboxamido)phenyl)acetate; (1R, 2S, 5R)-N-(2-cyanophenyl)-2-isopropyl-5-methylcyclohexane-carboxamide; and, (2S, 5R)-N-(3-cyanophenyl)-2-isopropyl-5-methylcyclohexane-carboxamide.
